(19) United States
(12) Patent Application Publication
(10) Pub. No.: US 2021/0275185 A1
(43) Pub. Date: Sep. 9, 2021

(54) Catheter System for Delivery of a Filling Body to an Aneurysmal Sac in a Body Lumen

(71) Applicant: Michel Marinus Petrus Johannes Reijnen, Arnhem (NL)

(72) Inventor: Michel Marinus Petrus Johannes Reijnen, Arnhem (NL)

(21) Appl. No.: 17/190,483

(22) Filed: Mar. 3, 2021

(30) Foreign Application Priority Data
Mar. 4, 2020 (EP) 20160882.5

(51) Int. Cl. A61B 17/12; B33Y 80/00

(52) U.S. Cl. CPC A61B 17/12113; A61B 17/12031; A61B 17/12195; A61B 2017/00867; B33Y 80/00; A61B 2017/1205; A61B 17/12186

(57) ABSTRACT

The invention relates to a catheter system for delivery of a filling body to an aneurysmal sac in a body lumen, which catheter system includes a substantially tubular shaft having a proximal end, a distal end and a guidewire lumen extending between the proximal and distal ends; a substantially tubular sheath defining an interior volume, coaxially positioned over the tubular shaft and slidable relative to the tubular shaft; a compressible filling body arranged in a compressed state in the interior volume between the sheath and the shaft near the distal end of the tubular shaft; and where the compressible filling body is expandable, when released from the sheath, to a porous filling body having a substantially cylindrical lumen and an outer surface having a main dimension perpendicular to the longitudinal axis of the cylindrical lumen, which main dimension is at least 1.5 times the diameter of the cylindrical lumen.

### CROSS-REFERENCE TO RELATED APPLICATION

This application claims priority to European Patent Application No. 20160882.5 filed Mar. 4, 2020, the disclosure of which is hereby incorporated by reference in its entirety.

### BACKGROUND OF THE INVENTION

### Field of the Invention

The invention relates to a catheter system for delivery of a filling body to an aneurysmal sac in a blood vessel, prior or in addition to treatment with an endoprosthesis.

### Description of Related Art

The causes of aneurysms are sometimes unknown but often they are related to atherosclerosis. Aneurysms may occur in any artery in the body, but there is a preference for the aorta, the iliac arteries and the popliteal arteries. Aneurysms may also be iatrogenic or caused by injury. An aneurysm occurs when part of an artery wall weakens, allowing it to balloon out or widen abnormally and can have a fusiform or saccular shape. Such a weakened artery wall can provide a severe health risk for the patient and typically needs to be resolved when a threshold diameter is reached. Other problems, besides the risk on rupture, may include thrombosis and distal embolization.

Typically, aneurysms are either treated by conventional surgery, using a prosthetic graft or with the endovascular placement of a stent-graft, also referred to as endoprosthesis or endograft, depending on the aneurysm anatomy and patient characteristics. It is known to fill an aneurysm with a thrombogenic wire causing a thrombus to be formed in the aneurysmal sac and strengthening the aneurysm therewith, such that the health risk is reduced.

U.S. Pat. No. 8,974,487 discloses to introduce a net shaped sac into the aneurysmal sac, filling the net shaped sac with fill members such that the aneurysmal sac is substantially filled, The remaining space can then be filled by thrombi.

These techniques are typically suitable for saccular aneurysms, or pseudoaneurysms, which have a small access opening through the feeding artery. However, a fusiform aneurysm is a widening of the blood vessel with no particular access opening. Filling the space of such a fusiform aneurysm will block the flow through the blood vessel.

If an endovascular strategy, usually referred to as Endovascular Aneurysm Repair, or EVAR, is chosen, typically an endovascular placed stent-graft is placed in such aneurysms, which takes away the pressure from the aneurysmal sac. However, leaks may occur, which will again pressurize the aneurysmal sac and could require surgical or endovascular re-intervention. Such leaks are called endoleaks and occur particularly after endovascular treatment of an aneurysm.

U.S. Pat. No. 5,769,882 proposes a solution for such fusiform aneurysms. The publication teaches to introduce a tubular element in the blood vessel at the position of the fusiform aneurysm and then inject a liquid sealant between the tubular element and the aneurysmal sac wall. This will reduce the chance on endoleaks.

The sealant could be partially curable. The liquid sealant could pose a problem when the tubular element does not seal sufficiently against the blood vessel wall and liquid sealant could enter the blood stream.

### SUMMARY OF THE INVENTION

It is an object of the invention to propose an alternative in which the above mentioned disadvantages are reduced or even removed.

This object is achieved by a catheter system for delivery of a filling body to an aneurysmal sac in a blood vessel, which catheter system comprises:
a substantially tubular shaft, having a proximal end, a distal end and a guidewire lumen extending between the proximal and distal ends;
a substantially tubular sheath defining an interior volume, coaxially positioned over the tubular shaft and slidable relative to the tubular shaft;
a compressible filling body arranged in a compressed state in the interior volume between the sheath and the shaft near the distal end of the tubular shaft;

wherein the compressible filling body is expandable, when released from the sheath, to a porous filling body having a lumen, preferably substantially cylindrical, and an outer surface having a main dimension perpendicular to the longitudinal axis of the lumen, which main dimension is at least 1.5 times the main dimension, perpendicular to the longitudinal axis, of the lumen.

The main dimension of the outer surface is considered the dimension, which characterized the shape of the cross-section perpendicular to the longitudinal axis of the lumen. So, for a cylindrical filling body, this main dimension is the outer diameter. Also, for a sphere or ellipse shaped outer surface, this main dimension is the diameter of the sphere. For a rectangular cross-section, it would be the largest diagonal and so on.

With the catheter system according to the invention, a porous filling body can be delivered through a blood vessel into the aneurysmal sac of an aneurysm, such that the aneurysmal sac is substantially filled. The lumen in the filling body allows for a pass-through opening, in which a conventional stent, stent-graft, or other (endo)prosthesis can be arranged.

Because the filling body is compressible, the filling can be arranged between the shaft and sheath of the catheter system and can be deployed in the aneurysmal sac by retracting the sheath relative to the shaft, such that the filling body is allowed to expand in flow lumen of the aneurysmal sac. This procedure could be performed step-wise whereby the distal portion is deployed first. This would enable repositioning. Distal safety wires, through the catheter, could be added to facilitate this.

In case the stent-graft is a bifurcated system the inner lumen of the filling body may be enriched by a cross-over catheter that enables pre-cannulation from the contralateral side, before deployment of the filling body so that during deployment guidewires are present from both outflow vessels that eventually will host the limbs of the stent-graft.

The porosity of the filling body contributes to the compressibility, but also promotes thrombus formation and organization inside and around the filling body and rapid remodeling of the aneurysm. If any endoleaks do occur, the leaking will be reduced or stopped by the thrombus forming in the aneurysmal sac.

If a type I endoleak occurs despite the arrangement of a stent or the like, the filling body will ensure, that such leakage will only fill the inner lumen of the invention, but no longer the aneurysmal sac and re-pressurizing of the aneurysmal sac will be prevented.

Avoidance of type II endoleaks, because the filling body fills the aneurysmal sac will avoid them, will lead to more shrinkage of the aneurysm, with a positive impact on follow-up regimes, the incidence of adverse events, the re-intervention rates and mortality.

In relation to type III endoleaks the filling body will provide lateral stability to the stent-graft and as such there is a lower risk on component separation or material fatigue.

Because of the arrangement of the filling body, migration of the stent-graft is unlikely, as the stent-graft is fitted in the lumen of the filling body, such that there is minimal space in lateral direction. This lateral stability will attribute to the proximal stability of the stent-graft, as distal migration can only occur with an increased angulation of the endograft in the aneurysmal sac.

In a preferred embodiment of the catheter system according to the invention, the outer surface is non-cylindrical, preferably bulbous or spheroid shaped.

In a preferred embodiment of the catheter system according to the invention, a scaffold or stent could be arranged in the lumen of the filling body. A stent can be compressed and be positioned into the lumen, when the filling body is compressed between the shaft and the sheath. When the filling body is deployed into the aneurysmal sac, the stent will also expand to a designed diameter to ensure a sufficient large lumen in the filling body for another stent, stent-graft, graft or prosthesis to be arranged therein.

In yet another preferred embodiment of the catheter system according to the invention, the material of the filling body has an open cell structure, preferably of a shape memory polymer or another memory material.

The open cell structure provides the porosity of the filling body. Furthermore, the use of a shape memory polymer ensures that the filling body will deploy from the compressed state to a designed expanded state, which will sufficiently fill the aneurysmal sac without causing too much stress onto the wall of the aneurysmal sac.

Even more preferred is a catheter system according to the invention, wherein the material of the filling body is a non-isotropic 3D printed foam with a structure similar to cancellous and cortical fibres. Such a structure allows for good compressibility and allows for good thrombogenic properties when expanded into the aneurysmal sac.

The filling body could have a solid structure. In addition or as an alternative, the filling body could be provided with a changing density in radial direction. It could also be a be a thin walled structure, with a central cavity, like a balloon or have a polar array of open tubular structures.

In another embodiment of the catheter system according to the invention the filling body is composed out of a plurality of thrombogenic wires arranged to a body core in which the lumen is provided.

In still a further embodiment of the catheter system according to the invention the diameter of the circumscribing circle of the non-cylindrical outer surface in a plane perpendicular to the longitudinal axis of the lumen is between 5 cm and 10 cm.

The bulbous shape of the outer surface of the filling body should more or less correspond to the lumen of the aneurysmal sac in order to properly fill the aneurysmal sac when expanded. By ensuring that the circumscribing circle of the non-cylindrical outer surface in a plane perpendicular to the longitudinal axis of the lumen is between 5 cm and 10 cm, the filling body would be suitable for most abdominal aneurysms. For aneurysm in other parts of the body, such as the thoracic aorta, the iliac artery and the popliteal artery, other dimensions would be required (2 cm to 10 cm). When used as an adjunct to avoid gutter formation in chimney-EVAR procedures the diameter would be even smaller (8 mm to 12 mm).

In still a further preferred embodiment of the catheter system according to the invention the filling body is coated with a hemostatic coating, such as fibrinogen, fibrin or collagen. The hemostatic coating further promotes thrombus forming around the porous structure of the filling body.

In yet another embodiment of the catheter system according to the invention the lumen of the filling body is flared on at least one, preferably both ends to facilitate cannulation.

Preferably, the filling body could be of a bioresorbable material, such that the filling body dissolves well after the thrombus is formed and organized and is no longer needed.

In order to be able to correctly position the filling body, the filling body could be made of a biodegradable material or a radiopaque material, such that it can be seen under X-ray illumination. It could also be provided with radiopaque markers on one or both longitudinal ends.

Furthermore, the guidewire lumen or an additional lumen in the tubular shaft could be used for delivering a contrast media or a drug, before the filling body is deployed.

The filling body, especially when made of a porous material, could additionally be loaded with a drug such that after the delivery of the filling body in the aneurysmal sac also a desired drug is present.

Furthermore, the filling body could be pre-loaded with a catheter that can be cannulated in a still compressed state from the contralateral side.

### BRIEF DESCRIPTION OF THE DRAWINGS

These and other features of the invention will be elucidated in conjunction with the accompanying drawings.

FIG. 1 shows a cross-sectional view of an embodiment of the catheter system according to the invention in a first state.

FIG. 2 shows the catheter system of FIG. 2 in a second state.

FIG. 3 shows a perspective view of the filling body of the catheter system according to FIG. 1.

FIG. 4 shows a cross-sectional view of the filling body of FIG. 3.

FIGS. 5A-5D show different steps of a method for deploying the catheter system of FIG. 1.

### DESCRIPTION OF THE INVENTION

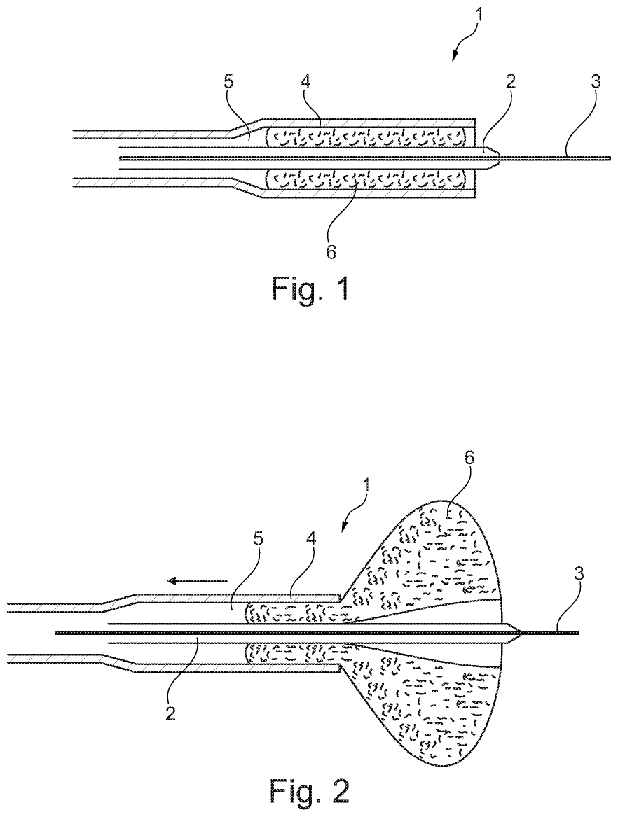

FIG. 1 shows a cross-sectional view of a catheter system 1 according to the invention. The catheter system 1 has a tubular shaft 2, through which a guide wire 3 extends. A tubular sheath 4 defining an interior volume 5 is coaxially positioned over the tubular shaft 2.

A compressible filling body 6 is arranged in a compressed state in the interior volume 5 near the distal end of the tubular shaft 2.

FIG. 2 shows the catheter system 1 in a partially deployed state. The sheath 4 is retracted relative to the shaft 2, such that the compressible filling body 6 is partially pushed out of the interior volume 5, such that the filling body 6 is allowed to expand.

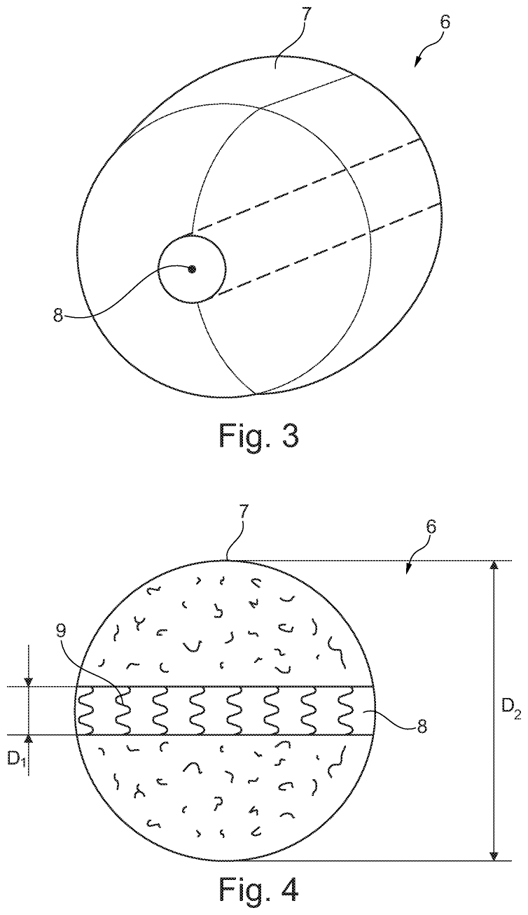

FIG. 3 shows the filling body 6 in fully expanded state. The filling body 6 has a bulbous outer surface 7 with a cylindrical lumen 8 extending through the filling body 6.

FIG. 4 shows a cross-sectional view of the filling body 6. A nitinol stent 9 is arranged in the cylindrical lumen 8, which has a diameter of D1. This nitinol stent 9 expands together with the porous filing body when the filling body 6 is moved out of the internal volume 5. The filling body 6 has a bulbous shape and a main dimension of D2, which is at least 1.5 times larger than the diameter D1.

Instead of a bulbous shape, the filling body could also have a more cylindrical or even fully cylindrical shape. Relevant is that there is sufficient material available between the cylindrical lumen and the outer surface, in which porous material thrombus forming and organization can occur in order to further fill the aneurysmal sac.

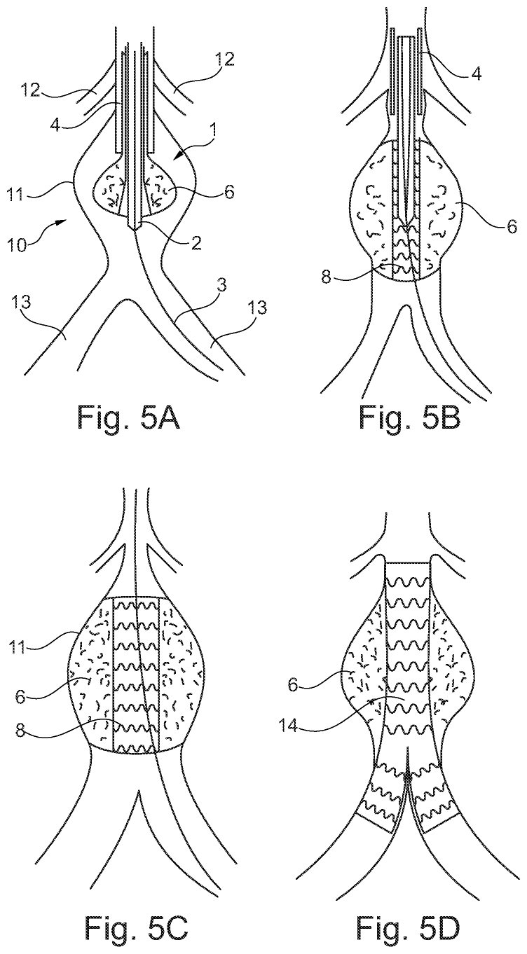

FIGS. 5A-5D show the abdominal aorta 10 with an abdominal aortic aneurysm 11, renal arteries 12 and common iliac arteries 13.

The catheter system 1 is introduced into the abdominal aorta 10 in a known way. The filling body 6 is pushed out of the internal volume 5, such that the filling body 6 expands into the abdominal aortic aneurysm 11. (see FIG. 5A en 5B). Upon expansion, the nitinol stent 8 also expands (see FIG. 5B) such that a cylindrical lumen is provided in the filling body 6. (see FIG. 5C)

The cylindrical lumen formed by the nitinol stent 8 allows for a pass-through opening through the abdominal aortic aneurysm 11, which is now completely filled with the porous filling body 6.

This pass-through opening can now be used to position a common prosthesis 14 into the abdominal aorta 10.

1. A catheter system for delivery of a filling body to an aneurysmal sac in a body lumen, which catheter system comprises:
a substantially tubular shaft having a proximal end, a distal end and a guidewire lumen extending between the proximal and distal ends;
a substantially tubular sheath defining an interior volume, coaxially positioned over the tubular shaft and slidable relative to the tubular shaft;
a compressible filling body arranged in a compressed state in the interior volume between the sheath and the shaft near the distal end of the tubular shaft;
wherein the compressible filling body is expandable, when released from the sheath, to a porous filling body having a lumen, preferably substantially cylindrical, and an outer surface having a main dimension perpendicular to the longitudinal axis of the lumen, which main dimension is at least 1.5 times the diameter of the main dimension, perpendicular to the longitudinal axis, of the lumen.

2. The catheter system according to claim 1, wherein the outer surface is non-cylindrical, preferably bulbous shaped, ellipsoidal or spheroid.

3. The catheter system according to claim 1, wherein a stent is arranged in the lumen of the filling body.

4. The catheter system according to claim 1, wherein the material of the filling body has an open cell structure, preferably of a shape memory polymer or other memory material.

5. The catheter system according to claim 4, wherein the material of the filling body is a non-isotropic 3D printed foam with a structure similar to cancellous and cortical fibres.

6. The catheter system according to claim 1, wherein the filling body is composed out of a plurality of thrombogenic wires arranged to a body core in which the substantially cylindrical lumen is provided.

7. The catheter system according to claim 1, wherein the diameter of the circumscribing circle of the non-cylindrical outer surface in a plane perpendicular to the longitudinal axis of the lumen is between 2 cm and 10 cm.

8. The catheter system according to claim 1, wherein the filling body is coated with a hemostatic coating, such as fibrinogen, fibrin or collagen or another coating promoting aneurysm sac remodeling.

9. The catheter system according to claim 1, wherein the lumen of the filling body is flared on at least one, preferably both ends to facilitate cannulation.
